(19) United States
(12) Patent Application Publication
(10) Pub. No.: US 2007/0015777 A1
(43) Pub. Date: Jan. 18, 2007

(54) Use of Inhibitors of the Ubiquitin Proteasome Pathway as a Method of Increasing Contractility of the Heart

(75) Inventors: Erik Bush, Erie, CO (US); Rick Gorczynski, Westminster, CO (US); Keith Koch, Erie, CO (US); Tim McKinsey, Broomfield, CO (US)

Correspondence Address: FULBRIGHT & JAWORSKI L.L.P., 600 CONGRESS AVE., SUITE 2400, AUSTIN, TX, 78701, US

(73) Assignee: Myogen, Inc.

(21) Appl. No.: 11/457,012

(22) Filed: Jul. 12, 2006

Related U.S. Application Data
(60) Provisional application No. 60/699,189, filed on Jul. 14, 2005.

(51) Int. Cl. A61K 31/519; A61K 31/4184; A61K 31/336

(52) U.S. Cl. 514/267; 514/381; 514/394; 514/475

(57) ABSTRACT

The present invention describes novel uses for inhibitors of the ubiquitin proteasome degradative pathway. In particular it describes methods for improving cardiac function, increasing alpha myosin levels in the heart, and increasing SERCA levels in the heart. The invention also provides methods for treating cardiac hypertrophy through inhibiton of the ubiquitin proteasome.

The present invention claims benefit of priority to U.S. Provisional Application Ser. No. 60/699,189, filed Jul. 14, 2005, the entire contents of which are hereby incorporated by reference.

### BACKGROUND OF THE INVENTION

1. Field of the Invention

The present invention relates generally to the fields of developmental biology and molecular biology. More particularly, it concerns gene regulation and cellular physiology in the heart and specifically in cardiomyocytes. More specifically, the invention relates to the use of inhibitors of the ubiquitin proteasome to enhance expression of alpha myosin heavy chain (α-MyHC) and smooth endoplasmic reticulum Ca²⁺ ATPase (SERCA). In particular, it relates to the use of such inhibitors to increase contractility in the heart and to treat a disease state where an increase in either contractility or α-MyHC expression would be beneficial.

2. Description of Related Art

The contractile proteins of the heart lie within the muscle cells, called myocytes, which constitute about 75% of the total volume of the myocardium. The two major contractile proteins are the thin actin filament and the thick myosin filament. Each myosin filament contains two heavy chains and four light chains. The bodies of the heavy chains are intertwined, and each heavy chain ends in a head. Each lobe of the bi-lobed myosin head has an ATP-binding pocket, which has in close proximity the myosin ATPase activity that breaks down ATP to its products.

The velocity of cardiac muscle contraction is controlled by the degree of ATPase activity in the head regions of the myosin molecules. The major determinant of myosin ATPase activity and, therefore, of the speed of muscle contraction, is the relative amount of the two myosin heavy chain isomers, α and β (MyHC). The α-MyHC isoform has approximately 2-3 times more enzymatic activity than the β-MyHC isoform and, consequently, the velocity of cardiac muscle shortening is related to the relative percentages of each isoform. For example, adult rodent ventricular myocardium has approximately 80-90% α-MyHC, and only 10-20% β-MyHC, which explains why its myosin ATPase activity is 3-4 times greater than bovine ventricular myocardium, which contains 80-90% β-MyHC.

In some models of heart disease, a change occurs in the expression of MyHC isoforms, with α-MyHC decreasing and β-MyHC increasing. These “isoform switches” reduce the contractility of the ventricle, ultimately leading to myocardial failure. This pattern of altered MyHC gene expression is a vivid in vivo reflection of the important role played by MyHC gene expression in controlling contractility of the heart.

Although the abnormalities in cardiac function which accompany heart disease may vary widely, decreased release from the sarcoplasmic reticulum (SR) of the Ca²⁺ ions required for activation of contractile proteins is a common characteristic of many cardiac syndromes. The significance of this loss can be best understood in the context of the role that calcium transport plays in the normal functioning of the heart.

Briefly, the SR is a membranous structure that surrounds each myofibril of cardiac muscle. SERCA is contained within the SR membranes and serves to actively transport 70 to 80% of free calcium ions into the SR intracellular space during diastolic relaxation of cardiac muscle. Much of the remaining calcium ions available for transport are removed from the cytoplasm by a SR sodium/calcium transport exchange system as well as, to a far lesser extent, transport driven by ATP hydrolysis catalyzed by sarcolemma calcium ion ATPase and through mitochondrial calcium uptake (Bassani et al., 1992; Carafoli, 1987).

Given that both the ATP hydrolytic activity of SERCA and the absolute levels of SERCA mRNA are decreased in the diseased heart (Hasenfuss et al., 1994; Studer et al., 1994), it has been widely postulated that the impairment of the heart's ability to receive blood at low pressures in disease states is directly linked to delays in SERCA mediated transport of contraction-activating calcium ions into the SR, which in turn results in a slowing of diastolic relaxation of the heart (see, e.g., Grossman, 1991; Lorell, 1991; Arai et al., 1994). These observations, particularly with respect to reductions in levels of mRNA's coding for SERCA have been confirmed in humans as well as other mammalian species (see, regarding human SERCA2 mRNA levels, Arai et al., 1994; and Mercadier et al., 1990; also, regarding lowering of SERCA2 mRNA levels in hypertrophied heart tissue of other mammalian species, see, e.g., Wang et al., 1994; Afzal and Dhella, 1992; Feldman et al., 1993).

Thus, there remains an unmet need to treat cardiovascular diseases such as hypertrophy, diastolic dysfunction and diseases related to contractile abnormalities in the heart associated with expression of α-MyHC and SERCA. α-MyHC and SERCA are in and of themselves targets for cardiovascular disease therapy, but the ubiquitin proteasome pathway, which as presented herein is involved in the degradation of MyHC and SERCA, represents a novel target for treating cardiac diseases or upregulating these specific genes.

### SUMMARY OF THE INVENTION

Thus, in accordance with the present invention, there is provided a method of improving the contractility of a heart comprising (a) identifying a subject or patient with aberrant or decreased contractility; and (b) treating said subject with an inhibitor of the ubiquitin proteasome pathway. The subject may be human or non-human. In specific embodiments of the invention, said inhibitor of the ubiquitin proteasome pathway may consist of MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG.

In yet other specific embodiments, improving contractility of the heart further comprises improving the diastolic function of the heart, or causing improvement in one or more symptoms of disease caused by decreased contractility of the heart. It is further contemplated that the symptoms that are improved may be shortness of breath or dyspnean upon exertion.

In yet other specific embodiments, improving contractility of the heart further comprises increasing the force of contraction of the heart or increasing the speed of relaxation of the heart.

In certain specific embodiments of the invention, identifying a heart with aberrant or decreased contractility comprises identifying a heart that has an abnormal ejection fraction or an abnormal left ventricular dp/dt.

In additional embodiments of the invention, it is contemplated that treatment will constitute treating said heart with at least one additional pharmaceutical agent. These additional pharmaceutical agents may be one or more of agents selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent. In other embodiments, the additional pharmaceutical agent may be selected from the group consisting of one or more of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor. In specific embodiments, it is contemplated that the additional pharmaceutical agent may be enoximone, ambrisentan, bosentan, sitaxsentan, or darusentan.

Also provided is a method of treating cardiac hypertrophy or heart failure comprising (a) identifying a subject or patient with cardiac hypertrophy or heart failure; and (b) treating said subject with an inhibitor of the ubiquitin proteasome pathway. The subject may be human or non-human. In specific embodiments of the invention, said inhibitor of the ubiquitin proteasome pathway may consist of MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG.

In specific embodiments, treating comprises improving one or more symptoms of cardiac hypertrophy. The one or more symptoms may comprise increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

In other embodiments, treating comprises improving one or more symptoms of heart failure, wherein said one or more symptoms comprises progressive remodeling, ventricular dilation, decreased cardiac output, impaired pump performance, arrhythmia, fibrosis, necrosis, energy starvation, and apoptosis.

In additional embodiments of the invention, it is contemplated that treatment will constitute treating said heart with at least one additional pharmaceutical agent. These additional pharmaceutical agents may be one or more of agents selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent. In other embodiments, the additional pharmaceutical agent may be selected from the group consisting of one or more of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor. In specific embodiments it is contemplated that the additional pharmaceutical agent may be enoximone, ambrisentan, bosentan, sitaxsentan, or darusentan.

In further embodiments, the method of treatment further comprises increasing the content of α-MyHC or SERCA in the heart of a subject. In certain specific embodiments, increasing the content of α-MyHC or SERCA consists of one or more of increasing the protein levels of x-MyHC or SERCA, increasing the RNA levels of α-MyHC or SERCA, or decreasing the rate of degradation of x-MyHC or SERCA.

In additional embodiments of the invention, it is contemplated that treatment will constitute treating said heart with at least one additional pharmaceutical agent. These additional pharmaceutical agents may be one or more of agents selected from the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent. In other embodiments, the additional pharmaceutical agent may be selected from the group consisting of one or more of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor. In specific embodiments, it is contemplated that the additional pharmaceutical agent may be enoximone, ambrisentan, bosentan, sitaxsentan, or darusentan.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—The proteasome inhibitor MG132 increases cardiac alpha myosin heavy chain protein expression in cultured cardiac myocytes. Cytoblot assay for alpha myosin heavy chain protein content in neonatal rat cardiac myocytes (NRVM) cultured in the absence or presence of MG132 (100 nM) for a period of 48 hours.

FIG. 2—The proteasome inhibitor MG132 suppresses hypertrophic agonist-dependent increases in cardiac beta myosin heavy chain protein expression. Cytoblot assay for β-myosin heavy chain protein content in NRVM cultured in the absence or presence of phenylephrine (20 μM) and MG132 (100 and 1000 nM) for a period of 48 hours.

FIG. 3—The proteasome inhibitor MG132 decreases expression of cardiac phospholamban and increases expression of SERCA, alpha myosin heavy chain and phospho-phospholamban. Western blot analysis of NRVM cultured in the absence or presence of phenylephrine (20 μM) and MG132 (370, 1500 and 3000 nM) for a period of 48 hours.

FIG. 4—The proteasome inhibitor MG132 suppresses hypertrophic agonist-dependent secretion of cardiac atrial natriuretic factor (ANF) at non-cytotoxic concentrations. Competitive ANF ELISA and cytotoxicity (adenylate kinase activity) assays performed on media supernatants recovered from NRVM cultured in the absence or presence of phenylephrine (20 μM) and a range of MG132 concentrations for a period of 48 hours. MG132 suppresses cardiac ANF secretion with an EC₅₀ of 20 nM.

FIG. 5—The proteasome inhibitor MG132 increases cardiac modulatory calcineurin interacting protein 1 (MCIP1) protein expression in cultured cardiac myocytes. Cytoblot assay for MCIP1 protein content in NRVM cultured in the presence of a range of MG132 concentrations for a period of 48 hours. MG132 induces cardiac MCIP1 expression with an EC₅₀ of 10 nM.

FIG. 6—The proteasome inhibitor MG132 increases cardiac modulatory calcineurin interacting protein 1 (MCIP1) protein expression and total ubiquitinated protein in cultured cardiac myocytes. Western blot analysis for MCIP1 protein and total ubiquitinated protein in NRVM cultured in the presence of a range of MG132 concentrations for a period of 48 hours. MG132 induces cardiac MCIP1 expression at concentrations that also induced accumulation of ubiquitinated protein.

FIG. 7—The proteasome inhibitor MG132 suppresses calcineurin-dependent nuclear import of the pro-hypertrophic transcription factor nuclear factor of activated T cells (NFAT). Immunofluorescence micrographs of NRVM infected with adenovirus encoding activated calcineurin and GFP-NFAT in the presence or absence of MG132 (1 μM).

### DESCRIPTION OF ILLUSTRATIVE EMBODIMENTS

Identifying new, more suitable candidates having the ability to modulate the fetal gene program or increase the contractility of cardiac tissue is an important goal of current research efforts. This led to the discovery, presented herein, that inhibitors of the ubiquitin proteasome degradative pathway are capable of upregulating α-MyHC, SERCA, and may be both cardiotonic and a treatment for cardiovascular diseases.

### I. UBIQUITIN PROTEASOME

The 26S proteasome is the multi-catalytic protease responsible for the majority of intracellular protein turnover in eukaryotic cells, including proteolytic degradation of damaged, oxidized or misfolded proteins, as well as processing or degradation of key regulatory proteins required for various cellular function (Ciechanover, 1994; Coux et al., 1995; Goldberg et al., 1995). Protein substrates are first marked for degradation by covalent conjugation to multiple molecules of a small protein, ubiquitin. The resultant polyubiquitinated protein is then recognized and degraded by the 26S proteasome.

Constituting the catalytic core of the 26S proteasome is the 20S proteasome, a multi-subunit complex of approximately 700 kDa molecular weight (Coux et al., 1995).

The ubiquitin-proteasome pathway plays a central role in a large number of physiological processes (Deshaies, 1995; Hoyt, 1997). The prior art teaches that regulated proteolysis of cell cycle proteins, including cyclins, cyclin-dependent kinase inhibitors, and tumor suppressor proteins, is required for controlled cell cycle progression and that proteolysis of these proteins occurs via the ubiquitin-proteasome pathway (Palombella et al., 1994), and WO 95/25533 teaches that activation of the transcription factor NF-κB, which itself plays a central role in the regulation of genes involved in the immune and inflammatory responses, is dependent upon the proteasome-mediated degradation of an inhibitory protein, IrB-α. WO 94/17816 discloses that the continual turnover of cellular proteins by the ubiquitin-proteasome pathway plays an essential role in antigen presentation.

While serving an essential physiological role, the ubiquitin-proteasome pathway also mediates the inappropriate or accelerated protein degradation that occurs as a result or cause of pathological conditions such as cancer, inflammatory diseases, or autoimmune diseases, in which these normal cellular processes have become deregulated. In addition, U.S. Pat. No. 5,340,736 teaches that the cachexia or muscle wasting associated with conditions such as cancer, chronic infectious diseases, fever, muscle disuse (atrophy), nerve injury, renal failure, and hepatic failure results from an increase in proteolytic degradation by the ubiquitin-proteasome pathway. Gonzales et al. (1996) teaches that the cytoskeletal reorganization that occurs during maturation of protozoan parasites is proteasome-dependent. It has also been shown that the ubiquitin-proteasome pathway is a target for treating ischemia and reperfusion injury, including preventing, reducing the size, or lessening the severity of infarcts following vascular occlusions such as occur during heart attack or stroke (U.S. Pat. No. 6,271,199).

The ubiquitin-proteasome system has also been implicated in the pathogenesis of cardiovascular diseases (reviewed by Hermann et al., 2004). In a hypothesis paper, Field and Clark (1997) speculated that altered or inappropriate ubiquitin conjugation of key regulatory proteins may contribute to the pathologic mechanism that underlies the transition from compensated cardiac hypertrophy to decompensated heart failure. Weekes et al. (2003) confirmed that human dilated cardiomyopathy (DCM) hearts possess elevated levels of ubiquitinated protein, and hypothesized that inappropriate ubiquitin conjugation may contribute to the DCM phenotype by causing the proteolysis of regulatory factors. It has also been shown that the ubiquitin-proteasome pathway is a target for treating ischemia and reperfusion injury, including preventing, reducing the size, or lessening the severity of infarcts following vascular occlusions such as occur during heart attack or stroke (U.S. Pat. No. 6,271,199). Finally, it has been shown in a cell culture model (using neonatal rat ventricular myocytes (NRVMs)) that use of the proteasome inhibitor MG132 is anti-hypertrophic as evidenced by measuring β-MyHC levels, cell size and morphology, brain natriuretic peptide (BNP), and levels of sarcomeric actin (Dreger, 2002).

Inhibition of proteasome activity thus offers a promising new approach for therapeutic intervention in these and other conditions directly or indirectly mediated by the proteolytic function of the proteasome.

### II. CARDIOVASCULAR DISEASE

Cardiovascular diseases are among the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

It follows that heart disease and its manifestations, including heart failure, DCM, and cardiac hypertrophy, presents a major health risk in the United States today. The cost to diagnose, treat and support patients suffering from these diseases is well into the billions of dollars. With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been fully elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Unfortunately, many of the commonly used agents have numerous adverse effects or are not fully effective. For example, certain diuretics may increase serum cholesterol and triglycerides. Moreover, diuretics are generally ineffective for patients suffering from severe heart failure. If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent monotherapy (i.e., a drug that improves cardiac output by increasing the force of myocardial muscle contraction) is associated with a panoply of adverse reactions, including gastrointestinal problems and central nervous system dysfunction.

Thus, the currently used pharmacological agents have severe shortcomings in particular patient populations. The availability of new, safe and effective agents would undoubtedly benefit patients who either cannot use the pharmacological modalities presently available, or who do not receive adequate relief from those modalities.

### III. TRANSCRIPTIONAL PATHWAY FOR HEART FAILURE OR CARDIAC HYPERTROPHY

It is known that Ca²⁺ activation is involved in a variety of forms of heart failure and heart disease. Ca²⁺ store depletion, or a raise in the cytoplasmic Ca²⁺ levels in the cell, has been show to stimulate a calcineurin dependent pathway for cardiac hypertrophy. This pathway is almost always (but perhaps not universally) involved in the hypertrophic response of the heart to the diverse stimuli that do lead to cardiac enlargement. Targeting agents that disrupt this pathway may be highly effective as methods of therapy or even as preventative methods to stop, ablate, or prevent cardiovascular disease. It is known that in cardiovascular diseases, α-MyHC and SERCA levels decrease, and the “fetal gene” program is activated. As such, reversing this process (“reverse remodeling”) is a key component of any treatment for heart failure or hypertrophy. Such reverse remodeling will yield increases in α-MyHC and SERCA, as well as inhibiting or modulating any one of the below mentioned members of the hypertrophy cellular pathway. The individual components of the pathway as they relate to cardiac hypertrophy and heart failure are discussed in further detail herein below.

A. Calcineurin

Calcineurin is a ubiquitously expressed serine/threonine phosphatase that exists as a heterodimer, comprised of a 59 kD calmodulin-binding catalytic A subunit and a 19 kD Ca²⁺-binding regulatory B subunit (Stemmer and Klee, 1994; Su et al., 1995). Calcineurin is uniquely suited to mediate the prolonged hypertrophic response of a cardiomyocyte to Ca²⁺ signaling because the enzyme is activated by a sustained Ca²⁺ plateau and is insensitive to transient Ca²⁺ fluxes as occur in response to cardiomyocytc contraction (Dolmetsch et al., 1997).

Activation of calcineurin is mediated by binding of Ca²⁺ and calmodulin to the regulatory and catalytic subunits, respectively. Previous studies showed that over-expression of calmodulin in the heart also results in hypertrophy, but the mechanism involved was not determined (Gruver et al, 1993). It is now clear that calmodulin acts through the calcineurin pathway to induce the hypertrophic response. Calcineurin has been shown previously to phosphorylate NF-AT3, which subsequently acts on the transcription factor MEF-2 (Olson and Williams, 2000). Once this event occurs, MEF-2 activates a variety of genes known as fetal genes, the activation of which inevitably results in hypertrophy.

CsA and FK-506 bind the immunophilins cyclophilin and FK-506-binding protein (FKBP12), respectively, forming complexes that bind the calcineurin catalytic subunit and inhibit its activity. CsA and FK-506 block the ability of cultured cardiomyocytes to undergo hypertrophy in response to AngII and PE. Both of these hypertrophic agonists have been shown to act by elevating intracellular Ca²⁺, which results in activation of the PKC and MAP kinase signaling pathways (Sadoshima et al., 1993; Sadoshima and Izumo, 1993; Kudoh et al., 1997; Yamazaki et al., 1997, Zou et al., 1996). CsA does not interfere with early signaling events at the cell membrane, such as PI turnover, Ca²⁺ mobilization, or PKC activation (Emmel et al., 1989). Thus, its ability to abrogate the hypertrophic responses of AngII and PE suggests that calcineurin activation is an essential step in the AngII and PE signal transduction pathways.

B. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al, 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

NF-AT3 is a 902-amino acid protein with a regulatory domain at its amino-terminus that mediates nuclear translocation and the Rel-homology domain near its carboxyl-terminus that mediates DNA binding. There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. Each step of the activation pathway may be blocked by CsA or FK506. This implies, and earlier studies have shown, that calcineurin is the protein responsible for NF-AT activation.

Thus, many of the changes in gene expression in response to calcineurin activation are mediated by members of the NF-AT family of transcription factors, which translocate to the nucleus following dephosphorylation by calcineurin. Many observations have supported the conclusion that NF-AT also is an important mediator of cardiac hypertrophy in response to calcineurin activation. NF-AT activity is induced by treatment of cardiomyocytes with AngII and PE. This induction is blocked by CsA and FK-506, indicating that it is calcineurin-dependent. NF-AT3 synergizes with GATA4 to activate the cardiac specific BNP promoter in cardiomyocytes. Also, expression of activated NF-AT3 in the heart is sufficient to bypass all upstream elements in the hypertrophic signaling pathway and evoke a hypertrophic response.

The crystal structure of the DNA binding region of NF-ATc has revealed that the C-terminal portion of the Rel-homology domain projects away from the DNA binding site and also mediates interaction with AP-1 in immune cells (Wolfe et al., 1997). According to the currently accepted model (Crabtree and Olson, 2002) hypertrophic stimuli such as AngII and PE, which lead to an elevation of intracellular Ca(2+), result in activation of calcineurin. NF-AT3 within the cytoplasm is dephosphorylated by calcineurin, enabling it to translocate to the nucleus where it can interact with GATA4, and then activate the transcription factor MEF-2, a family of transcription factors that are normally repressed by a tight association with class II HDAC's. Calcineurin activation of NF-AT3 regulates hypertrophy in response to virtually all pathologic stimuli.

C. MEF-2

As mentioned above, NF-AT3 activation by Calcineurin leads to the activation of another family of transcription factors, the monocyte enhancer factor-2 family (MEF-2), which are known to play an important role in morphogenesis and myogenesis of skeletal, cardiac, and smooth muscle cells (Olson et al., 1995). MEF-2 factors are expressed in all developing muscle cell types, binding a conserved DNA sequence in the control regions of the majority of muscle-specific genes. Of the four mammalian MEF-2 genes, three (MEF-2A, MEF-2B and MEF-2C) can be alternatively spliced, which have significant functional differences (Brand, 1997; Olson et al., 1995). These transcription factors share homology in an N-terminal MADS-box and an adjacent motif known as the MEF-2 domain. Together, these regions of MEF-2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF-2 factors regulate myogenesis through combinatorial interactions with other transcription factors.

Loss-of-function studies indicate that MEF-2 factors are essential for activation of muscle gene expression during embryogenesis. The expression and functions of MEF-2 proteins are subject to multiple forms of positive and negative regulation, serving to fine-tune the diverse transcriptional circuits in which the MEF-2 factors participate. MEF-2 is bound in an inactive form in the healthy heart by class II HDACS (see supra), and when MEF-2 is activated it is released from the HDAC and activates the fetal gene program that is so deleterious for the heart. Thus MEF-2 is a far downstream modulator of hypertrophy, but it is a key modulator in that it appears to mediate (along with Class II HDACs) the transcription of the fetal genes that are always reactivated when the heart undergoes pathologic remodeling.

D. Histone Deacetylase

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive. It was thus a novel finding and highly relevant to treatment of heart disease that HDAC's were shown to interact with MEF-2 and that HDAC's play a significant role in the control of the fetal gene program (see U.S. Pat. No. 6,706,686 hereinafter incorporated in its entirely by reference).

No less than 17 different HDACs have been cloned from vertebrate organisms. The first three human HDACs identified were HDAC 1, HDAC 2 and HDAC 3 (termed class I human HDACs), and HDAC 8 (Van den Wyngaert et al., 2000) was later added to the list of Class I HDACs. Later, class II human HDACs, HDAC 4, HDAC 5, HDAC 6, HDAC 7, HDAC 9, and HDAC 10 (Kao et al., 2000) were cloned and identified (Grozinger et al., 1999; Zhou et al. 2001; Tong et al., 2002). Additionally, HDAC 11 was identified (Gao et al., 2002), leading to the labeling of a third class of HDACs, Class III HDACs (Thiagalingam et al., 2003). All HDACs appear to share homology in the catalytic region. HDACs 4, 5, 7, 9 and 10 however, have a unique amino-terminal extension not found in class I HDACs. This amino-terminal region contains the MEF-2-binding domain. HDACs 4, 5, 7 and 9 have been shown to be involved in the regulation of cardiac gene expression and in particular embodiments, repressing MEF-2 transcriptional activity. The exact mechanism in which class II HDAC's repress MEF-2 activity is not completely understood. One possibility is that HDAC binding to MEF-2 inhibits MEF-2 transcriptional activity, either competitively or by destabilizing the native, transcriptionally active MEF-2 conformation. It also is possible that class II HDAC's require dimerization with MEF-2 to localize or position HDAC in a proximity to histones for deacetylation to proceed.

A variety of inhibitors for histone deacetylase have been identified. The proposed uses range widely, but primarily focus on cancer therapy. See Saunders et al. (1999); Jung et al. (1997); Jung et al. (1999); Vigushin et al. (1999); Kim et al. (1999); Kitazomo et al. (2001); Vigusin et al. (2001); Hoffmann et al. (2001); Kramer et al. (2001); Massa et al. (2001); Komatsu et al. (2001); Han et al. (2000). Such therapy is the subject of NIH sponsored clinical trials for solid and hematological tumors. HDAC's also increase transcription of transgenes, thus constituting a possible adjunct to gene therapy. (Yamano et al., 2000; Su et al., 2000). Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al., 1996). It is commercially available from a variety of sources including BIOMOL Research Labs, Inc., Plymouth Meeting, Pa. A substantial listing of available HDAC inhibitors can be found in U.S. Pat. No. 6,706,686 and World Patent Application WO 04/112763 hereinafter incorporated by reference.

E. MCIP

Another gene that is associated with heart failure and hypertrophy, primarily due to its tight association with and regulation by calcineurin, is the human gene (DSCR1) encoding MCIP 1, one of 50-100 genes that reside within a critical region of chromosome 21 (Fuentes et al, 1997; Fuentes et al., 1995), trisomy of which gives rise to the complex developmental abnormalities of Down syndrome, which include cardiac abnormalities and skeletal muscle hypotonia as prominent features (Epstein, 1995). MCIP (called ZAKI-4) was identified from a human fibroblast cell line in a screen for genes that are transcriptionally activated in response to thyroid hormone (Miyazaki et al., 1996).

MCIP1 directly binds and inhibits calcineurin, functioning as an endogenous feedback inhibitor of calcineurin activity. Overexpression of MCIP1 in the hearts of transgenic animals is anti-hypertrophic; MCIP1 attenuates in vivo models of both calcineurin-dependent hypertrophy (Rothermel et al., 2001) and pressure-overload-induced hypertrophy (Hill et al., 2002). MCIP1 also acts as a substrate for phosphoryalation by MAPK and GSK-3, and calcineurin's phosphatase activity.

Binding of MCIP1 to calcineurin does not require calmodulin, nor does MCIP interfere with calmodulin binding to calcineurin. This suggests that the surface of calcineurin to which MCIP1 binds does not include the calmodulin binding domain. In contrast, the interaction of MCIP1 with calcineurin is disrupted by FK506:FKBP or cyclosporin:cyclophylin, indicating that the surface of calcineurin to which MCIP1 binds overlaps with that required for the activity of immunosuppressive drugs.

MCIP, as well as all the aforementioned genes, each in and of themselves present enticing therapeutic targets for heart failure and hypertrophy. A major reason for the inventors' interest in inhibiting the ubiquitin proteasome is that MCIP and α-MyHC may be turned over via this pathway. As such, treatment of heart failure or hypertrophy by inhibitin the proteasome would represent a major leap forward over the current methods available for treating patients suffering from cardiovascular diseases.

### IV. α-MyHC AND SERCA

A. α-MyHC

Myosin is present in all muscle and non-muscle cells. Of the ten distinct classes of myosin in human cells, myosin-II is the form responsible for contraction of skeletal, cardiac, and smooth muscle. This form of myosin is significantly different in 20 amino acid composition and in overall structure from myosins in the other nine; distinct classes (Goodson and Spudich, 1993). Myosin-II consists of two globular head domains, called Subfragment-1 or S 1, linked together by a long-helical coiled coiled tail. Proteolysis of myosin generates either S 1 or heavy meromyosin (HMM, a two-headed form with a truncated tail), depending on conditions.

Although myosin isoforms from various tissues differ in a number of biological properties, they all share the same basic molecular structure as a dimer of two heavy chains (approximately 200 kDa) noncovlantly associated with two pairs of light chains (approximately 20 and 17 kDa). The two globular amino-terminal heads are tethered together by the carboxyl-terminal alpha-helical coiled-coil that forms a tail. The tails are involved in the assembly of myosin molecules into filaments, whereas the heads contain an actin-activated Mg²⁺-ATPase activity. Each myosin head can be divided by three protease-sensitive regions into peptides of approximately 20, 25, 50 kDa.

The myosin molecule contains an alpha-helical coiled-coiled tail involved in self assembly of myosin molecules into bipolar thick filaments. These thick filaments interdigitate between thinner actin filaments and the two filament systems slide past one another during contraction of the muscle. This filament sliding mechanism involves conformational changes in the myosin heads causing them to walk along the thin actin filaments at the expense of ATP hydrolysis.

MyHC has been studied at the molecular level in striated muscle. MyHC contains an amino-terminal motor or head domain, a neck that is the site of light-chain binding, and a carboxy-terminal tail domain. Conventional myosins, such as those found in muscle tissue, are composed of two myosin heavy-chain subunits, each associated with two light-chain subunits that bind at the neck region and play a regulatory role. Unconventional myosins, believed to function in intracellular motion, may contain either one or two heavy chains and their associated light chains. There is evidence for about 25 myosin heavy chain genes in vertebrates, more than half of them unconventional.

The heavy myosin chain head domain ends in an amino acid sequence which is conserved in most myosins. The neck domains of most MyHC consist of a variable number of motifs with a conserved sequence believed to be the site for light-chain binding. Calmodulin or calmodulin-like proteins function as light chains. An unexpected degree of variation has been observed in the tail domains of different myosins. Several unconventional myosins contain domains associated with signal transduction (Mooseker et al., 1995).

Disorders of myosin function are involved in a variety of human diseases including muscle disorders, developmental disorders, and cancer. Two forms of myosin heavy chain (α and β) have been observed in the mammalian ventricular myocardium. The speed with which the heart contracts is related to their relative expression, with greater contractile speed seen in hearts of species that have higher amounts of α-MyHC compared to the β form. These findings suggested that increased α-MyHC expression may be therapeutic in cardiovascular disease. Mutations in genes coding for the β-MyHC have been related to hypertrophic cardiomyopathy (Marian, et al., 1998). It is widely accepted in the art that decreases in α-MyHC are associated with decreased contractility, decreased cardiac performance, and is a hallmark of hypertrophy when it is coupled with an increase in β-MyHC (Abraham et al., 2002; Lowes et al., 2002; Miyata et al., 2000). Thus, agents that can increase the content of α-MyHC in the cell or prevent its rapid turnover could be useful for improving cardiac function or treating cardiovascular disease.

B. SERCA

SERCA, like α-MyHC, is involved in regulating the function of the heart. The sarcoplasmic reticulum (SR) is an internal membrane system, which plays a critical role in the regulation of cytosolic Ca²⁺ concentrations and thus, excitation-contraction coupling in muscle. In cardiac cells release of Ca²⁺ from the SR leads to contraction whereas in smooth muscle cells it induces vasorelaxation through activation of Ca²⁺ activated potassium channels and hyperpolarisation of the cell.

Since Ca²⁺ acts as a major intracellular messenger, elevating these levels affects a wide range of cellular processes including contraction, secretion and cell cycling (Dawson, 1990; Evans et al., 1991). Three genes code for the different isoforms of SERCA that are known in vertebrates, SERCAla/b, SERCA2a/b and SERCA3. The SERCA isoforms are usually tagged to the endoplasmic reticulum (ER) or ER subdomains like the sarcoplasmic reticulum, although the precise subcellular location is often not known. The SERCA proteins belong to the group of ATP-driven ion-motive ATPases, which also includes, amongst others, the plasma membrane Ca²⁺ transport ATPases (PMCA), the Na(⁺)K(⁺)-ATPases, and the gastric H(⁺)K(⁺)ATPases. The SERCA Ca²⁺ transport ATPases can be distinguished from their plasma membrane counterparts like PMCA by the specific SERCA inhibitors: thapsigargin, cyclopiazonic acid, and 2,5-di(tert-butyl)-1,4-benzohydroquinone (Thastrup et al., 1990; Seidler et al., 1989; Oldershaw and Taylor, 1990).

SERCA2 is expressed in muscle and non-muscle cells. Cardiac muscle expresses 5- to 20-fold higher levels of SERCA2 than smooth muscle. Slow-twitch skeletal and cardiac muscle only express SERCA2a, while SERCA2b (referred to as the “housekeeping” isoform) is expressed in all non-muscle tissue, and represents about 75% of the Ca²⁺ transporting ATPase activity in smooth-muscle tissue.

In slow-twitch skeletal muscle, cardiac muscle and smooth-muscle tissues, SERCA2 activity is modulated by phosphorylation of the regulatory protein phospholamban (PLB) (see Fuji et al., 1991). In cardiac muscle, in vivo phosphorylation of PLB by cAMP- or Calcium/Calmodulin-dependent protein kinase has a positive effect on the Ca²⁺ transport (Le Peuch et al., 1997; Tada et al., 1979; Davis et al., 1983; Wegener et al., 1989). In order to determine the exact in vivo role of phospholamban, PLB-deficient mice have been generated (Luo et al., 1994). A marked effect is observed on Ca²⁺ uptake, whereas no effect is measured in Vmax. The ablation of the PLB gene in mice is associated with increased myocardial contractility, and a loss of the positive inotropic response to β-adrenergic stimulation. The precise molecular mechanism underlying the modulation of SERCA by PLB is not apparent. An electrostatic mechanism has been proposed, as a direct interaction between PLB and SERCA, in which the unphosphorylated PLB inhibits the SERCA pump (Chiesi and Schwaller, 1999), but it is not the only model proposed to explain the modulation of SERCA by PLB (see, e.g., Voss et al., 1994). Direct phosphorylation of SERCA by Ca²⁺/CaM kinase II results in a 2-fold higher maximal velocity (Xu and Kirchberger, 1999). This CaM kinase phosphorylation is specific for SERCA2 and may act synergistically with the phosphorylation of phospholamban.

Thus, it can be seen that SERCA plays an important role in regulating calcium levels, and hence in pathologies related to abnormal Ca²⁺ concentrations and regulation. For instance, major pathologies in which SERCA may play a role include cardiac hypertrophy, heart failure, and hypertension (Arai et al., 1994; Lompre et al., 1994). In animal models of cardiac hypertrophy, where most studies are performed, a highly significant positive correlation has been obtained between end-diastolic cytosolic Ca²⁺ levels and diastolic relaxation abnormalities. After aortic binding, SERCA2 mRNA and protein levels are decreased, as is the sarcoplasmic reticulum Ca²⁺ uptake (Komuro et al., 1989; de la Bastie et al, 1990). This effect was only found in cases of severfe hypertrophy, and was only observed when heart failure occurred. In moderate hypertrophy and in cases of compensated hypertrophy, no changes in the level of SERCA mRNA were observed (de la Bastie et al., 1990).

In humans, most studies report a decrease in SERCA2 mRNA, SERCA2 protein levels and decreased Ca²⁺ uptake in a failing heart (Arai et al., 1993; Hasenfuss et al., 1994). The decreased levels of SERCA2 expression are accompanied by decreased expression of PLB, cardiac ryanodine receptor and dihydropyridine receptor (Vatner et al., 1994; Go et al., 1995; Takahashi et al., 1992). These human heart failure data are confirmed in different animal models. In hypertrophic animals, SERCA2 expression levels are decreased; in a dilated strain of animals, Ca²⁺ uptake is decreased with increasing age (Kuo et al., 1992). Most striking in both humans and animal models is the strong positive correlation between SERCA2 and PLB mRNA levels. Cardiac hypertrophy as well as calcium's deleterious effects on contractile function can be prevented by activating SR Ca²⁺ uptake either by SERCA2a gene transfer or by decreasing the inhibitory effect of phospholamban on SERCA2a (reviewed in Kaprielian et al., 2002). Thus, as mentioned previously for α-MyHC, agents that can increase the content of SERCA in the cell or prevent its rapid turnover could be useful for improving cardiac function (i.e., improving contractility) or treating cardiovascular diseases like hypertrophy or heart failure.

### V. CONTRACTILITY AND DIASTOLIC DYSFUNCTION

A. Contractility

The cardiac contractile apparatus is comprised of functional units called sarcomeres, which contain both thick and thin filaments. The thick filament is made up of 300 individual myosin molecules, as well as structural proteins such as C-protein, α-actinin, β-actinin, M-line proteins, C-protein, and titin. Myosin is the most abundant of all of the muscle proteins, constituting 15-30% of the total protein, and is arranged in such a way that the head regions protrude at right angles from the body such that they can interact with actin molecules. The thin filaments are ropelike, and are essentially a polymer of actin molecules that coil around each other in a double helical array. Tropomyosin (Tm) lies in the groove of the actin helices and stretches along the actin filament. It makes contact with 7 actins and a nearby Tm. Troponin (Tn) complexes are found at regular intervals along the thin filament, and are comprised of three subunits. Troponin-T (TnT) links the whole Tn complex to Tm, and Tn-I (I for inhibitory) blocks the interaction between actin and myosin until Ca²⁺ binds to Tn-C(C for calcium) and removes the inhibitory effect of Tn-I.

Contraction is mediated by the two main contractile proteins, actin and myosin, and occurs when actin and myosin filaments slide past each other. Myosin crossbridges extend from the body of myosin towards the actin filament, and the repeated attachment and detachment of crossbridges occurs in cycles. During each cycle, ATP is hydrolyzed and an oarlike motion is produced that drives the actin filament along the myosin. Regulation of this cycle is quite complex, but involves interactions between Ca²⁺, thin filament regulatory proteins (Tm and Tn), and myosin crossbridges. Current models propose that thin filaments exist in three distinct states, depending on the relative position of Tm. In the absence of Ca²⁺, thin filaments are in a “blocked” state in which Tm prevents myosin from interacting strongly with actin. Once Ca²⁺ becomes available and binds to TnC, the Tn complex changes conformation and the thin filament undergoes a transition to the “closed” state. In this state, increased numbers of weakly bound myosin crossbridges are able to interact with actin. While in this closed state, some of the weakly bound crossbridges become strongly bound but are not yet able to produce force. However, this non force-generating state promotes further movement of Tm into an “open” state. In the open state, strongly bound crossbridges become force-generating and contraction occurs. According to this model, complete activation of thin filaments occurs only in the presence of Ca(2+) and strongly bound myosin crossbridges. Ca²⁺ binding initiates the events and allows crossbridges to form, which in turn promotes additional crossbridge formation. As mentioned before, α-MyHC exhibits 2-3 times the myofibrillar ATPase activity and actin filament sliding velocity as P-MyHC. Therefore, myocyte preparations containing exclusively α-MyHC show 2-3 fold higher ATPase activity and velocity of contraction than β-MyHC expressing tissues. These differences in functional activity result in increased power output and stroke volume, and ultimately enhance cardiac performance.

The importance of the sarcomeric proteins in maintaining normal cardiac function is underscored by the large number of FHCs in which mutations in thick or thin filament proteins have been found. FHCs are a group of inherited, autosomal dominant diseases that cause hypertrophic cardiomyopathy (Dalloz et al., 2001). This group of diseases is fairly common, affecting around 1 in 500 individuals and representing a major cause of sudden death in otherwise healthy young adults. More than 60 different mutations have been identified in the P-MyHC gene alone, accounting for 30-40% of all of the documented human cases of FHC (Dalloz et al., 2001). Most of these mutations reside in the head or motor domain of β-MyHC. Mutations have also been identified in the ventricular essential and regulatory myosin light chain, cardiac TnT, cardiac TnI, α-Tm, C-protein, actin, and titin. However, it is likely that other genetic components will be identified and shown to contribute to this group of diseases.

The precise molecular mechanisms underlying the disease process are not yet known, but one common feature is that mutations in different sarcomeric proteins lead to the same phenotype. This suggests that the pathogenesis of the disease is similar in this family of diseases, and that the mutations have a common set of functional consequences. In vitro studies analyzing the function of mutated sarcomeric proteins are beginning to shed some light on the mechanisms underlying the functional defects, and in vivo studies are now underway using transgenic animal models that recapitulate the human disease. Collectively, these studies should provide insight into the complex nature of FHCs.

The rate of increase in ventricular pressure, denoted as +dP/dtₘₐₓ, is commonly used as an index of ventricular contractility. Therefore it is a measure that can be used to analyze the efficacy (or lack thereof) of a compound or agent that inhibits the ubiquitin proteasome as a method for improving cardiac function or contractility. Another measure of cardiac performance is ejection fraction (EF). During each heartbeat, the heart contracts and relaxes. When the heart contracts (systole), it ejects blood from the pumping chambers (ventricles). When the heart relaxes (diastole), the ventricles refill with blood. No matter how forceful the contraction, it doesn't empty all of the blood out of a ventricle. The term ejection fraction refers to the percentage of blood that is pumped out of a filled ventricle with each heartbeat. This measures the capacity at which the heart is pumping. Because the left ventricle is the heart's main pumping chamber, EF is usually measured in the left ventricle. A normal EF is 55 percent to 70 percent. The EF may decrease when the heart muscle has been damaged, due to a disease or disorder or even an injury. Therefore, as with +dP/dtₘₐₓ, a compound or agent that is capable of normalizing or improving the EF would be a useful therapeutic, and compounds that alter (x-MyHC or SERCA levels have the capacity to act in this manner. Thus, the methods of the present invention seek to improve the EF and cause physiologically beneficial increases in α-MyHC and SERCA by inhibiting the ubiquitin proteasome.

B. Diastolic Dysfunction

Ventricular function is highly dependent upon preload. Therefore, if ventricular filling (preload) is impaired, this will lead to a decrease in stroke volume. The term “diastolic dysfunction” refers to changes in ventricular diastolic properties that have an adverse effect on stroke volume (for a complete review of the topic see Lippincott, 2004, hereinafter incorporated in full by reference).

Ventricular filling (i.e., end-diastolic volume and hence sarcomere length) depends upon the venous return and the compliance of the ventricle during diastole. A reduction in ventricular compliance, as occurs in ventricular hypertrophy, will result in less ventricular filling (decreased end-diastolic volume) and a greater end-diastolic pressure (and pulmonary capillary wedge pressures) as shown to the right by changes in the ventricular pressure-volume loop. Stroke volume, therefore, will decrease. Depending on the relative change in stroke volume and end-diastolic volume, there may or may not be a small decrease in ejection fraction. Because stroke volume is decreased, there will also be a decrease in ventricular stroke work.

A second mechanism can also contribute to diastolic dysfunction: impaired ventricular relaxation (reduced lusitropy). Near the end of the cycle of excitation-contraction coupling in the myocyte, the sarcoplasmic reticulum actively sequesters Ca²⁺ so that the concentration of Ca²⁺ in the vicinity of troponin-C is reduced allowing the Ca²⁺ to leave its binding sites on the troponin-C and thereby permit disengagement of actin from myosin. This is a necessary step to achieve rapid and complete relaxation of the myocyte. If this mechanism is impaired (e.g., by reduced rate of Ca²⁺ uptake by the sarcoplasmic reticulum), or by other mechanisms that contribute to myocyte relaxation, then the rate and perhaps the extent of relaxation are decreased. This will reduce the rate of ventricular filling, particularly during the phase of rapid filling.

An important and deleterious consequence of diastolic dysfunction is the rise in end-diastolic pressure. If the left ventricle is involved, then left atrial and pulmonary venous pressures will also rise. This can lead to pulmonary congestion and edema, often manifested physically as shortness of breath. If the right ventricle is in diastolic failure, the increase in end-diastolic pressure will be reflected back into the right atrium and systemic venous vasculature. This can lead to peripheral edema and ascites. Thus, agents that can reduce the ventricular pressure found in diastolic dysfunction could act to improve systemic blood pressure, thereby alleviating the shortness of breath and peripheral (and pulmonary) edema found in this disorder.

### VI. METHODS OF TREATING

A. Proteasome Inhibitors

Inhibitors of the ubiquitin proteasome (IUP's) have been described in the literature. Non-limiting examples that may be useful in the present invention include MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG. The following publications, hereinafter incorporated by reference, also disclosue IUP's that may be used in accordance with the methods of the present invention: Tawa et al. (1997); PCT WO 05/025515; PCT WO 05/021558; PCT WO 99/46298; PCT WO 99/37666; PCT WO 99/15183; PCT WO 98/13061; PCT WO 98/28436; U.S. Pat. No. 6,096,711.

B. Combined Therapy

In another embodiment, it is envisioned to use an IUP in combination with other therapeutic modalities, both as dual or combination therapy with 3 or more therapeutic agents. Thus, in addition to the use of an IUP, one may also provide more “standard” pharmaceutical therapies as adjunct or additional therapy. Examples of other therapies include, without limitation, “beta blockers,” anti-hypertensives, cardiotonics, anti-thrombotics, vasodilators and prostenoids, hormone antagonists, iontropes, diuretics, endothelin receptor antagonists (ERA's or ETRA's), calcium channel blockers, phosphodiesterase inhibitors, ACE inhibitors, angiotensin type 2 antagonists and cytokine blockers/inhibitors, HDAC inhibitors, TRP Channel inhibitors, and 5-HT2 receptor modulators.

Combinations may be achieved by administering a single composition or pharmacological formulation that includes both agents, or by co-administering two distinct compositions or formulations, at the same time, at different times, in the same or different formulations. Alternatively, the therapy using an IUP may precede or follow administration of the other agent(s) by intervals ranging from minutes to weeks. In embodiments where the other agent(s) are applied separately, one would generally ensure that a significant period of time did not expire between the time of each delivery, such that the additionally agent would still be able to exert an advantageously combined effect on the patient. In such instances, it is contemplated that one would typically administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an IUP or the other agent will be desired. In this regard, various combinations may be employed. By way of illustration, where the IUP is “A” and the other agent is “B,” the following permutations based on 3 and 4 total administrations are exemplary:

A/B/A B/A/B B/B/A A/A/B B/A/A A/B/B B/B/B/A B/B/A/
B A/A/B/B A/B/A/B A/B/B/A B/B/A/A B/A/B/A B/A/A/B
B/B/B/A A/A/A/B B/A/A/A A/B/A/A A/A/B/A A/B/B/B B/
A/B/B B/B/A/B

Other combinations are likewise contemplated.

D. Adjunct Therapeutic Agents

Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention are listed below, and may include an antihyperlipoproteinemic agent, an antiarteriosclerotic agent, an antithrombotic/fibrinolytic agent, a blood coagulant, an antiarrhythmic agent, an antihypertensive agent, a vasopressor, a vasodilator, a prostenoid, a treatment agent for congestive heart failure, an antianginal agent, an antibacterial agent or a combination thereof.

1. Antihyperlipoproteinemics

In certain embodiments, administration of an agent that lowers the concentration of one of more blood lipids and/or lipoproteins, known herein as an “antihyperlipoproteinemic,” may be combined with the therapy according to the present invention. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, camitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a b-adrenergic blocker, a b-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, endothelin receptor antagonists, phosphodiesterase inhibitors, vasodilators and miscellaneous antihypertensives.

a. Alpha Blockers

Non-limiting examples of an alpha blocker, also known as an a-adrenergic blocker or an a-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a J3-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a B3-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, beraprost, chloracizine, chromonar, clobenfurol, clonitrate, dilazep, dipyridamole, droprenilamine, efloxate, erythrityl tetranitrane, etafenone, fendiline, flolan, floredil, ganglefene, herestrol bis(b-diethylaminoethyl ether), hexobendine, iloprost, itramin tosylate, khellin, lidoflanine, mannitol hexanitrane, medibazine, nicorglycerin, pentaerythritol tetranitrate, pentrinitrol, perhexiline, pimethylline, prostenoids, prostacyclin, remodulin, trapidil, tricromyl, trimetazidine, troInitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ambrisentan, ajmaline, g aminobutyric acid, bosentan, bufeniode, cicletainine, ciclosidomine, a cryptenamine tannate, darusentan, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sildenafil, sitaxsentan, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quantemary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticmafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitors include amrinone (inocor), milrinone, and enoximone.

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

E. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures The previously mentioned formulations are all contemplated for treating patients suffering from heart failure or hypertrophy.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### VII. DEFINITIONS

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the prevention, improvement and/or reversal of symptoms of a specific disease, disorder, syndrome or state (i.e., improving the ability of the heart to pump blood in a heart failure setting). Improvement in the physiologic function of the heart may be assessed using any of the measurements described herein (e.g., measurement of ejection fraction, fractional shortening, left ventricular internal dimension, heart rate, etc.), as well as any effect upon the animal's survival. A compound which causes an improvement in any parameter associated with a specific disease used in the screening methods of the instant invention may thereby be identified as a therapeutic compound.

The terms “compound” and “chemical agent” may refer to any chemical entity, pharmaceutical, drug, protein, antibody, nucleic acid and the like that can be used to treat or prevent a disease, illness, sickness, or disorder of bodily function. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to a wide variety of pathophysiological, chemical, external and biological stresses through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “modulator” refers to any agent that is capable of altering the expression, stability, activity, efficacy, or potency of the proteasome. Modulators may include proteins, nucleic acids, carbohydrates, peptides, small molecules, antibodies, or any other molecule(s) which binds or interacts with a cellular or intracellular receptor, molecule, and/or pathway of interest. Modulators need not act directly on the proteasome, but may cause an upregulation of expression or activity or function (at the RNA or protein level) indirectly, via an effect on some other gene or protein that leads to alteration of the activity of the proteasome.

As used herein, the term “modulate” refers to a change or an alteration in a biological or chemical activity. Modulation may be an increase or a decrease in protein activity, a change in kinase activity, a change in binding characteristics, or any other change in the biological, functional, or immunological properties associated with the activity of a protein or other structure of interest.

As used herein, the term “select” or “selection” in the context of an inhibitor or modulator will be understood to mean making a choice between known or experimental compounds and agents that are capable of inhibiting or modulating the proteasome.

As used herein, the term “small molecule” refers to an organic molecule or its salt(s), usually having a molecular weight less than 1000 Daltons.

### VIII. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1

### Materials and Methods

NRVM culture. For preparations of neonatal rat ventricular myocytes (NRVMs), hearts were removed from 10-20 newborn (1-2 days old) Sprague-Dawley rats. Isolated ventricles were pooled, minced and dispersed by three 20-minute incubations at 37° C. in Ads buffer (116 mM NaCl, 20 mM HEPES, 10 mM NaH2PO4, 5.5 mM glucose, 5 mM KCl, 0.8 mM MgSO4, pH 7.4) containing collagenase Type 11 (65 units/ml, Worthington) and pancreatin (0.6 mg/ml, GibcoBRL). Dispersed cells were applied to a discontinuous gradient of 40.5% and 58.5% (v/v) Percoll (Amersham Biosciences), centrifuged, and myocytes collected from the interface layer. Myocyte preparations were pre-plated in Dulbecco's modified Eagle's medium (DMEM, Cellgro), supplemented with 10% (v/v) charcoal stripped fetal bovine serum (FBS, HyClone), 4 mM L-glutamine and 1% penicillin/streptomycin for 1 hour at 37° C. to reduce fibroblast contamination, then plated at a density of 2.5×10⁵ cells per well on 6-well tissue culture plates (or 10,000 cells/well on 96-well tissue culture plates) coated with a 0.2% (w/v) gelatin solution. After 24 hours in culture, myocyte preparations were transferred to serum-free maintenance medium (DMEM supplemented with 0.1% (v/v) Nutridoma (Roche), L-glutamine and penicillin/streptomycin). Where indicated, NRVM were treated with test compounds for a period of 48 hrs. For infection with calcineurin and GFP-NFAT adenovirus, NRVM were exposed to adenovirus at a multiplicity of infection (MOI) of 10 for 48 hrs. prior to analysis.

NRVM protein quantitation by cytoblot (alpha myosin, beta myosin, MCIP1). NRVM were plated overnight in 96-well plates. The next day, medium was replaced with serum-free maintenance medium for 4 hours, and test compounds added. Forty eight hours later, wells were washed twice with 100 μl/well PBS, aspirating between washes. Cells were fixed by adding 100 μl/well methanol for 30 min. Methanol was aspirated and wells washed twice with 100 μl/well PBS. Next, 100 μl/well blocking solution (PBS+1% BSA) was added for 1 hr at room temperature. Blocking solution was aspirated and 50 μl/well primary antibody solution added in 1% BSA (α- or β-myosin hybridoma supernatant or MCIP1 polyclonal) for 1 hr at room temperature. Primary antibody solution was removed and wells washed three times with 100 μl/well PBS+1% BSA. Wash was aspirated and 50 μl/well secondary antibody solution added (1:500 dilution of goat anti-rabbit HRP conjugate in PBS+1% BSA; Southern Biotech #4050-05) for 1 hr at room temperature. Secondary antibody solution was removed and wells washed three times with 100 μl/well PBS. Wash was aspirated and 50 μl/well luminol solution added (Pierce #34080). Plates were read in a 96-well luminometer (Packard Fusion).

Western Blots. For protein sample preparation, cultured cells were lysed in extraction buffer (50 mM Tris, pH 7.5, 150 mM NaCl, 1% Triton X-100, 0.5% deoxycholic acid, 0.1% SDS) supplemented with protease inhibitors (1 mM AEBSF, 10 μg/ml aprotinin, 0.1 mM leupeptin, 2 mM EDTA). Homogenates were centrifuged 10 min at 4° C. at 16,000×g and supernatants recovered. Protein concentrations were determined by the bicinchoninic acid method (BCA Protein Assay, Pierce) with bovine serum albumin as a standard. Equivalent quantities of protein samples (10 μg/lane) were denatured in Laemmli buffer and resolved on Tris-glycine SDS-PAGE gels (4-20% acrylamide gradient, Invitrogen). Resolved proteins were transferred to nitrocellulose membranes, blocked in 5% nonfat dry milk, and incubated overnight with primary antibody (diluted in TBST; 50 mM Tris, pH 7.5, 150 mM NaCl, 0.1% Tween-20) supplemented with 5% nonfat dry milk. Membranes were washed, probed with horseradish peroxidase-conjugated secondary antibody and processed for enhanced chemiluminescence (SuperSignal reagent, Pierce).

ANF and toxicity assays. ANF in media supernatants was quantitated by competitive ELISA using a monoclonal anti-ANF antibody (Biodesign) and a biotinylated ANF peptide (Phoenix Peptide). Total cellular protein was quantitated by standard Coomassie dye-binding assay; cells were lysed in protein assay reagent (BioRad) and absorbance at A595 was measured after 1 hour. Cytotoxicity was quantitated by measuring release of adenylate kinase (AK) from cultured NRVM into culture medium (ToxiLight kit, Cambrex).

### B. Example 2

### Results

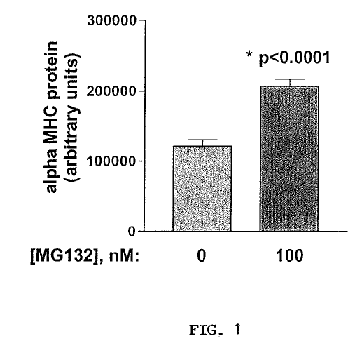

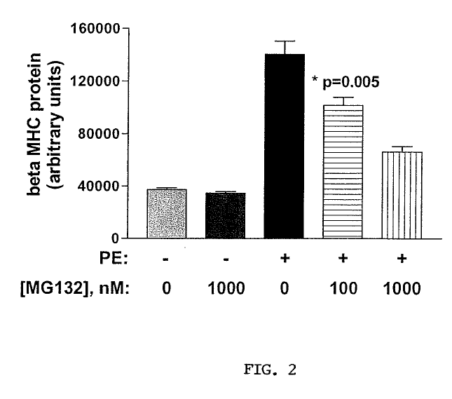

The proteasome inhibitor MG132 increases cardiac alpha myosin heavy chain protein and decreases beta myosin heavy chain protein in cultured cardiac myocytes. The inventors observed that exposure of cultured cardiac myocytes to MG132 significantly increased expression of alpha myosin heavy chain protein (FIG. 1). In contrast, MG132 treatment effectively suppressed adrenergic agonist (phenylephrine) dependent upregulation of beta myosin heavy chain protein (FIG. 2).

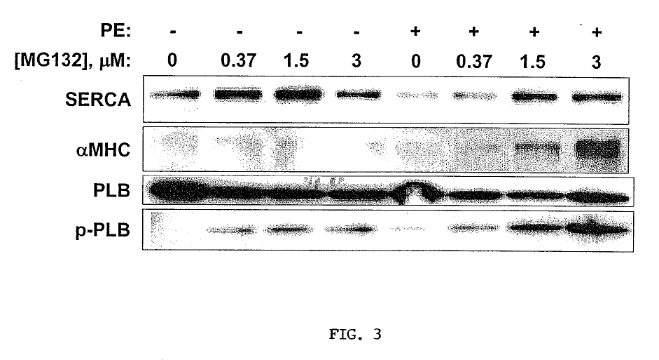

The proteasome inhibitor MG132 alters expression of key components of cardiomyocyte calcium handling and contractility. The inventors examined the effects of MG132 on the expression of other molecular markers linked to pathologic hypertrophy and contractile impairment (FIG. 3). MG132 increased in vitro expression of cardiac SERCA protein, and decreased expression of the SERCA inhibitory protein, phospholamban (PLB). MG132 also caused a dose-dependent increase in PLB phosphorylation, a post-translational modification that reduces the activity of PLB. Taken together, the observed effects of proteasome inhibition on the expression of myosin heavy chain isoforms and calcium handling proteins would predict enhanced cardiomyocyte contractility.

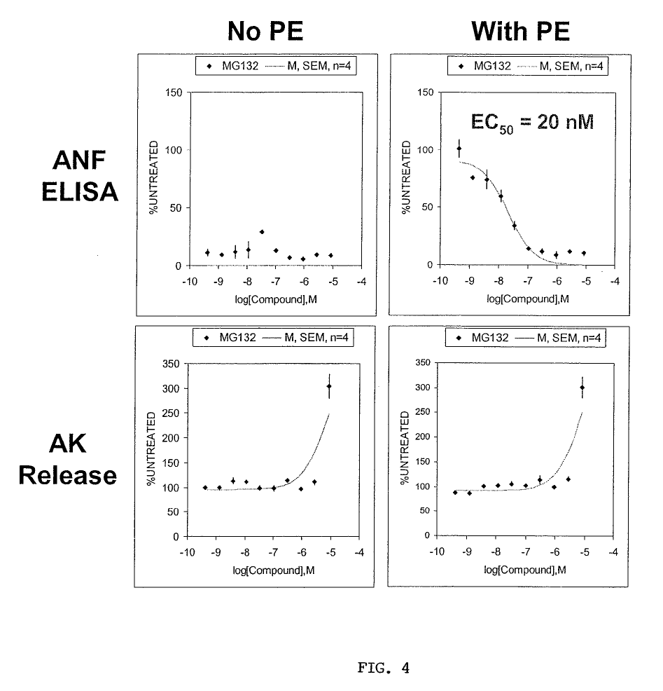

MG132 blocks agonist-dependent cardiomyocyte hypertrophy. The inventors examined the effects of proteasome inhibition on the ability of the hypertrophic agonist phenylephrine (PE) to induce cardiomyocyte hypertrophy, as measured by secretion of atrial natriuretic factor (ANF, a key index of hypertrophy). As shown in FIG. 4, MG132 exhibited dose-dependent suppression of PE-induced ANF secretion, with an EC50 of approximately 20 nM. Critically, suppression of hypertrophy occurred at concentrations of MG132 that produced no release of adenylate kinase (AK), a marker of cytotoxicity.

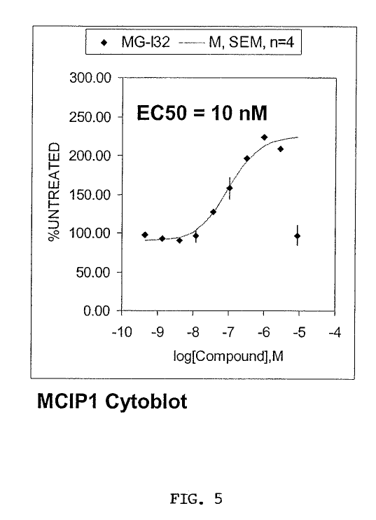

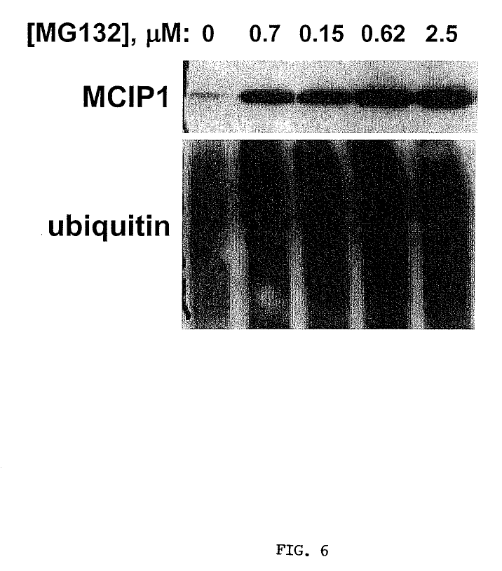

MG132 increases abundance of the endogenous calcineurin inhibitor MCIP1. The inventors observed that MG132 induced a dose-dependent increase in expression of modulatory calcineurin interacting protein 1 (MCIP1), an endogenous regulator of the pro-hypertrophic phosphatase calcineurin (FIG. 5). These observations were confirmed by Western blot analysis of MG132-treated cardiomyocytes (FIG. 6).

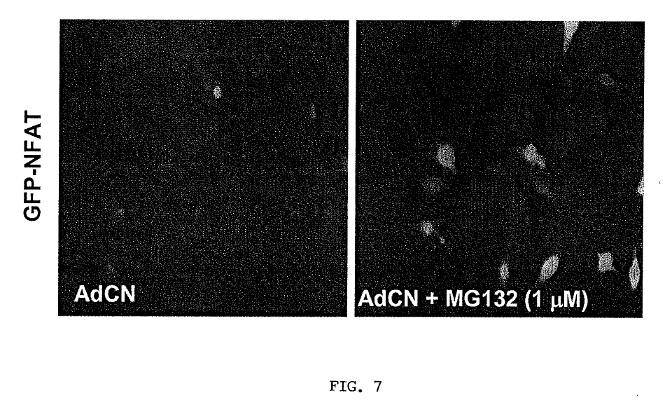

MG132 blocks calcineurin-dependent nuclear import of the pro-hypertrophic transcription factor NFAT. The transcription factor nuclear factor of activated T cells (NFAT) has been shown to be a downstream effector of the calcineurin signaling pathway. In response to hypertrophic stress signals activated cardiac calcineurin dephosphorylates cytosolic NFAT, which transits to the nucleus and facilitates hypertrophic gene expression. The inventors observed that MG132 blocked the ability of constitutively activated calcineurin to promote nuclear import of NFAT tagged with green fluorescent protein (FIG. 7), consistent with suppression of the calcineurin signaling pathway.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
Abraham et al., Mol. Med., 11:750-60 (2002).
Afzal and Dhella, Am. J. Physiol., 262:H868-H874 (1992).
Arai, et al., Circ. Res., 74:555-564 (1994).
Arai et al., Circ. Res., 72:463-469 (1993).
Bassani, et al., J. Physiol. 453:591-608 (1992).
Brand et al., J. Biochem. Cell. Biol., 29:1467-1470 (1997).
Carafoli, E., Ann. Rev. Biochem., 56:395-433 (1987).
Chiesi and Schwaller, FEBS Lett., 244:241-244 (1999).
Ciechanover, Cell, 79: 13-21 (1994).
Coux et al., Ann. Rev. Biochem., 65:801-847 (1995).
Crabtree, G. R. and Olson, E., Cell, 109:S67-S79 (2002).
Dalloz et al., J. Mol. Cell. Cardiol., 1:9-25 (2001).
Davis et al., J. Biol. Chem., 258:13587-13591 (1983).
Dawson, Essays Biochem., 25:1-37 (1990).
de la Bastie et al., Circ. Res., 66:554-564 (1990).
Deshaies, Trends Cell. Biol., 5: 428-434 (1995).
Dolmetsch et al., Nature, 386:855-858 (1997).
Dreger H., Thesis Submission to Humboldt University of Berlin, November 2002.
Durand et al., Ann. Med., 27:311-317 (1995).
Emmel et al., Science, 246:1617-1620 (1989).
Epstein, In The Metabolic and Molecular Bases of Inherited Disease: in Down Syndrome (Trisomy 21), Scriver, Beaudet, Valle, (eds), 7th Ed., Vol. 1, pp. 749-794, McGraw-Hill, Inc., New York, 1995.
Evans et al., J. Exp. Botany, 42:285-303 (1991).
Feldman, et al., Circ. Res., 73:184-192 (1993).
Field and Clark, Cardiovasc. Res., 33:8-12 (1997).
Fuentes et al., Genomics, 44:358-361 (1997).
Fuentes et al., Hum. Mol. Genet., 4:1935-1944 (1995).
Fuji et al., FEBS Lett., 273:232-234 (1991).
Gao et al., J. Biol. Chem., 277:25748-55 (2002).
Go et al., J. Clin. Invest., 95:888-894 (1995).
Goldberg et al., Chemistry & Biology, 2:503-508 (1995).
Goodman and Gilman's “The Pharmacological Basis of Therapeutics.”
Goodson and Spudich, Proc. Natl. Acad. Sci. U.S.A., 90(2):659-63 (1993).
Grossman, W., N. Engl. J. Med., 325:1557-1564 (1991).
Grozinger et al., Proc. Natl. Acad. Sci. USA, 96:4868-4873 (1999).
Gruver et al., Endocrinology, 133:376-388 (1993).
Han et al., Cancer Research, 60:6068-6074 (2000).
Hasenfuss, et al., Circ. Res., 75:434-442, (1994).
Hermann et al., Cardiovasc. Res., 61:11-21 (2004).
Hill et al., J. Biol. Chem., 277:10251-10255 (2002).
Ho et al., J. Biol. Chem., 270:19898-19907 (1995).
Hoffmann et al., Bioconjugate Chem., 12:51-55 (2001).
Hoey et al., Immunity, 2:461-472 (1995).
Hoyt, Cell, 91:149-151 (1997).
Jung et al., J. Med. Chem., 42:4669-4679 (1999).
Jung et al., Med. Chem. Lett., 7:1655-1658 (1997).
Kao et al., Genes Dev., 14:55-66 (2000).
Kaprielian et al., Basic Res. Cardiol., 97 Suppl 1:1136-45 (2002).
Kim et al., Oncogene, 18:2461-2470 (1999).
Kitazomo et al., J Clinical Endoc. Metabol., 86(7):3430-3435 (2001).
Komastsu et al., Cancer Res., 61:4459-4466 (2001).
Komuro et al., J Clin Invest., 83:1102-1108 (1989).
Kramer et al., Trends in Endoc. Metabolism, 12)₇):294-300 (2001).
Kudoh et al., Circ. Res., 80:139-146 (1997).
Kuo et al., Biochem. Biophys. ACTA, 1138:343-349 (1992).
Le Peuch et al., Biochemistry, 18:5150-5157 (1997).
Lippincott et al., Cardiovascular Physiology Concepts (2004).
Lompre et al., J. Mol. Cell. Cardiol., 26:1109-1121 (1994).
Lorell, B. H., Ann. Rev. Med., 42:411-436 (1991).
Lowes et al., N. Engl. J. Med., 18:1357-65 (2002).
Luo et al., Circ. Res., 75:401-409 (1994).
Marian et al., J Cardiovasc. Electrophysiol., 9:88-99 (1998).
Massa et al., J. Med. Chem., 44:2069-2072 (2001).
Masuda et al., Mol. Cell. Biol., 15:2697-2706 (1995).
McCaffery et al., Science, 262:750-754 (1993).
Mercadier, et al., J. Clin. Invest., 85:305-309 (1990).
Merck Index, Thirteenth Edition.
Miyata e al., Circ. Res., 4:386-90 (2000).
Miyazaki et al., J Biol. Chem., 271:14567-14571 (1996).
Mooseker et al., Annu. Rev. Cell Dev. Biol., 11:633-67 (1995).
Northrop et al., Nature, 369:497-502 (1994).
Oldershaw and Taylor, FEBS Lett., 274:214-216 (1990).
Olson and Williams, Cell, 101:689-692 (2000).
Olson and Williams, Bioassays, 22:510-519 (2000).
Olson et al., Dev. Biol., 172(1):2-14 (1995).
Palombella et al., Cell, 5:773-85 (1994).
Park et al., J. Biol. Chem., 271:20914-20921 (1996).
PCT Application No. WO 94/17816
PCT Application No. WO 95/25533
PCT Application No. WO 98/13061
PCT Application No. WO 98/28436
PCT Application No. WO 99/15183
PCT Application No. WO 99/32619
PCT Application No. WO 99/37666
PCT Application No. WO 99/46298
PCT Application No. WO 04/112763
PCT Application No. WO 05/25515
PCT Application No. WO 05/21558
Physicians Desk Reference.
Remington's Pharmaceutical Sciences.
Rooney et al., EMBO J., 13:625-633 (1994).
Rothermel et al., Proc. Natl. Acad. Sci. USA, 98:3328-3333 (2001).
Sadoshima et al, Cell, 75:977-984 (1993)
Sadoshima and Izumo, Circ. Res., 73:424-438 (1993).
Saunders et al., Cancer Res., 59-399-409 (1999).
Seidler et al., J. Biol. Chem., 264:17816-17823 (1989).
Stemmer and Klee, Biochemistry, 33:6859-6866 (1994).
Studer, et al., Circ. Res., 75:443-453 (1994).
Su et al., Cancer Res., 60:3137-3142 (2000).
Su et al., Eur. J. Biochem., 230:469-474 (1995).
Tada et al., J. Biol. Chem., 254: 319-326 (1979).
Takahashi et al., Antibiotics, 49:453 (1996).
Takahashi et al., J. Clin. Invest., 90:927-935 (1992).
Taunton et al., Science, 272:371 (1996).
Tawa et al., J. Clin. Invest., 100:197 (1997).
Thastrup et al., Proc. Natl. Acad. Sci., 87:2466-2477 (1990).
Thiagalingam et al., Ann. N.Y. Acad. Sci., 983:84-100 (2003).
Tong et al., Nucleic Acids Res., 30:1114-23 (2002).
U.S. Pat. No. 6,706,686
U.S. Pat. No. 6,271,199
U.S. Pat. No. 6,096,711
U.S. Pat. No. 5,708,158
U.S. Pat. No. 5,340,736
U.S. Pat. No. 4,265,874
U.S. Pat. No. 4,256,108
U.S. Pat. No. 4,166,452
Van den Wyngaert et al., FEBS Lett., 478:77-83 (2000).
Vatner et al., Circulation, 90:1423-1430 (1994).
Vigushin et al., Clinical Cancer Res., 7:971-976 (2001).
Vigushin et al., Cancer Res., 5S (1999).
Voss et al., Biophys. J., 67:190-196 (1994).
Wang, et al, Am. J. Physiol., 267:H918-H924 (1994).
Wegener et al, J. Biol. Chem., 264:11468-11474 (1989).
Weekes et al., Proteomics, 3:208-216 (2003).
Wolfe et al., Nature, 385:172-176 (1997).
Workman and Kingston, Annu. Rev. Biochem., 67:545-579 (1998).
Xu and Kirchberger, J. Biol. Chem., 264:16644-16651 (1999)
Yamazaki et al., Circulation, 95:1260-1268 (1997).
Yamano et al., Amer. Soc. Gene Ther. (2000).
Young et al., Handbook of Applied Therapeutics, 7.1-7.12 and 9.1-9.10 (1989).
Zhou et al., Proc. Natl. Acad. Sci., 98:10572-10577 (2001).
Zou et al., J. Biol. Chem., 271:33592-33597 (1996).

What is claimed is:

1. A method for improving the contractility of a heart in a subject comprising:
(a) identifying a subject having a heart with aberrant or decreased contractility of the heart; and
(b) treating said subject with an inhibitor of the ubiquitin proteasome pathway.

2. The method of claim 1, wherein the subject is a non-human subject.

3. The method of claim 1, wherein the subject is a human subject.

4. The method of claim 1, wherein improving the contractility comprises improving the diastolic function of the heart.

5. The method of claim 1, wherein improving the contractility comprises improving one or more symptoms of disease caused by decreased contractility of the heart.

6. The method of claim 5, wherein said one or more symptoms consists of shortness of breath in the patient or dyspnea upon exertion.

7. The method of claim 1, wherein improving the contractility comprises increasing the force of contraction of the heart.

8. The method of claim 1, wherein improving the contractility comprises increasing the speed of relaxation of the heart.

9. The method of claim 1, wherein identifying a subject with aberrant or decreased heart contractility comprises identifying a heart that has an abnormal ejection fraction or an abnormal left ventricular dp/dt.

10. The method of claim 1, wherein said inhibitor of the ubiquitin proteasome pathway consists of MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG

11. The method of claim 1, further comprising treating said heart with at least one additional pharmaceutical agent.

12. The method of claim 11, wherein said additional pharmaceutical agent is one or more of the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent.

13. The method of claim 11, wherein said additional pharmaceutical agent may be one or more of the group consisting of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor.

14. The method of claim 11, wherein said additional pharmaceutical agent may be enoximone, ambrisentan, bosentan, sitaxsentan or darusentan.

15. A method for treating cardiac hypertrophy or heart failure in a subject comprising:
(a) identifying a subject having cardiac hypertrophy and/or heart failure; and
(b) providing to said subject an inhibitor of the ubiquitin proteasome pathway.

16. The method of claim 15, wherein treating comprises improving one or more symptoms of cardiac hypertrophy.

17. The method of claim 16, wherein said one or more symptoms comprises increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

18. The method of claim 15, wherein treating comprises improving one or more symptoms of heart failure.

19. The method of claim 18, wherein one or more symptoms comprises progressive remodeling, ventricular dilation, decreased cardiac output, impaired pump performance, arrhythmia, fibrosis, necrosis, energy starvation, and apoptosis.

20. The method of claim 15, wherein said inhibitor of the ubiquitin proteasome pathway consists of MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG

21. The method of claim 15, further comprising treating said heart with at least one additional pharmaceutical agent.

22. The method of claim 21, wherein said additional pharmaceutical agent is one or more of the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent.

23. The method of claim 21, wherein said additional pharmaceutical agent may be one or more of the group consisting of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor.

24. The method of claim 21, wherein said additional pharmaceutical agent may be enoximone, ambrisentan, bosentan, sitaxsentan or darusentan.

25. A method for increasing the alpha myosin heavy chain (α-MyHC) content in the heart of a subject comprising administering to said subject an inhibitor of the ubiquitin proteasome pathway.

26. The method of claim 25, wherein increasing o-MyHC content comprises increasing the protein levels of α-MyHC, increasing the RNA levels of α-MyHC, or decreasing the rate of degradation of α-MyHC.

27. The method of claim 25, wherein said inhibitor of the ubiquitin proteasome pathway consists of MG132, PS1, MG262, PS341, PS273, lactacystin, P-lactone, NLVS, YLVS, dihydroeponemycin, epoxomicin, YU101, PS519, DFLB, MG115, TMC95A, gliotoxin, EGCG

28. The method of claim 25, further comprising treating said heart with at least one additional pharmaceutical agent.

29. The method of claim 28, wherein said additional pharmaceutical agent is one or more of the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent.

30. The method of claim 28, wherein said additional pharmaceutical agent may be one or more of the group consisting of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor.

31. The method of claim 28, wherein said additional pharmaceutical agent may be enoximone, ambrisentan, bosentan, sitaxsentan or darusentan.

32. A method for increasing the smooth endoplasmic reticulum Ca²⁺ ATPase (SERCA) content in the heart of a subject comprising administering to said subject an inhibitor of the ubiquitin proteasome pathway.

33. The method of claim 32, wherein increasing SERCA content comprises increasing the protein levels of SERCA, increasing the RNA levels of SERCA, or decreasing the rate of degradation of SERCA.

34. The method of claim 32, wherein said inhibitor of the ubiquitin proteasome pathway consists of MG132, PS1, MG262, PS341, PS273, lactacystin, β-lactone, NLVS, YLVS, dihydroeponemycin, epoxomicin, YU101, PS519, DFLB, MG115, TMC95A, gliotoxin, EGCG

35. The method of claim 32, further comprising treating said heart with at least one additional pharmaceutical agent.

36. The method of claim 35, wherein said additional pharmaceutical agent is one or more of the group consisting of an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent.

37. The method of claim 35, wherein said additional pharmaceutical agent may be one or more of the group consisting of a beta blocker, an inotrope, a diuretic, ACE-I, AII antagonist, BNP, a Ca²⁺-channel blocker, a phosphodiesterase inhibitor, an endothelin receptor antagonist, or an HDAC inhibitor.

38. The method of claim 35, wherein said additional pharmaceutical agent may be enoximone, ambrisentan, bosentan, sitaxsentan or darusentan.
